(19) United States
(12) Patent Application Publication
(10) Pub. No.: US 2026/0020772 A1
(43) Pub. Date: Jan. 22, 2026

(54) MAGNETIC RESONANCE DATA ACQUISITION APPARATUS, MAGNETIC RESONANCE DATA ACQUISITION METHOD, AND NON-TRANSITORY COMPUTER READABLE MEDIUM

(71) Applicant: Canon Medical Systems Corporation, Otawara-shi (JP)

(72) Inventors: Hidenori TAKESHIMA, Tokyo (JP); Shuki MARUYAMA, Nasushiobara (JP)

(73) Assignee: Canon Medical Systems Corporation, Otawara-shi (JP)

(21) Appl. No.: 19/199,551

(22) Filed: May 6, 2025

(30) Foreign Application Priority Data
Jul. 18, 2024 (JP) 2024-114742

(51) Int. Cl. A61B 5/055; G06V 10/25

(52) U.S. Cl. CPC A61B 5/055; G06V 10/25

(57) ABSTRACT

According to one embodiment, a magnetic resonance data acquisition apparatus includes processing circuitry. The processing circuitry is configured to obtain a designated region of interest. The processing circuitry is configured to generate a pulse sequence for acquiring magnetic resonance data multiple times based on the region of interest while changing at least one of a region for acquisition or a condition of setting a saturation pulse. The processing circuitry is configured to acquire multiple pieces of magnetic resonance data according to the pulse sequence.

### CROSS-REFERENCE TO RELATED APPLICATIONS

This application is based upon and claims the benefit of priority from Japanese Patent Application No. 2024-114742, filed Jul. 18, 2024, the entire contents of which are incorporated herein by reference.

### FIELD

Embodiments described herein relate generally to a magnetic resonance data acquisition apparatus, a magnetic resonance data acquisition method, and a magnetic resonance data acquisition program.

### BACKGROUND

In the acquisition of a magnetic resonance spectroscopy signal, a fat signal, if mixed in the signal, makes it difficult to analyze the signal. Thus, methods such as suppressing a fat signal band using saturation pulses, etc., suppressing a region likely to have fat using saturation pulses, etc., or adjusting a volume of interest (VOI) so as to exclude a region likely to have fat from the VOI, can be adopted.

However, in the method of suppressing a fat signal band using saturation pulses, the suppression affects not only the fat signal but also a spectrum of a desired metabolite signal. Also, in the method of suppressing a region likely to have fat or the method of adjusting the VOI so as to exclude a region likely to have fat from the VOI, fine adjustment needs to be made manually, causing variations in the precision depending on experience and capability.

### BRIEF DESCRIPTION OF THE DRAWINGS

FIG. 1 is a block diagram showing a magnetic resonance data acquisition apparatus according to an embodiment.

FIG. 2 is a flowchart showing an example of an operation of the magnetic resonance data acquisition apparatus.

FIG. 3 is a flowchart showing a detailed example of an operation performed by the magnetic resonance data acquisition apparatus with an acquisition method that includes a first method.

FIG. 4 is a diagram showing an example of setting a region of interest using the first method.

FIG. 5 is a flowchart showing a detailed example of an operation performed by the magnetic resonance data acquisition apparatus with an acquisition method that includes a second method.

FIG. 6 is a diagram showing an example of setting a pattern of change in saturation pulse according to the second method.

FIG. 7 is a diagram showing an example of displaying spectra of multiple MRS signals based on the acquisition method with the first method.

FIG. 8 is a diagram showing an example of displaying spectra of multiple MRS signals based on the acquisition method with the second method.

### DETAILED DESCRIPTION

In general, according to one embodiment, a magnetic resonance data acquisition apparatus includes processing circuitry. The processing circuitry is configured to obtain a designated region of interest. The processing circuitry is configured to generate a pulse sequence for acquiring magnetic resonance data multiple times based on the region of interest while changing at least one of a region for acquisition or a condition of setting a saturation pulse. The processing circuitry is configured to acquire multiple pieces of magnetic resonance data according to the pulse sequence.

Hereinafter, a magnetic resonance data acquisition apparatus, a magnetic resonance data acquisition method, and a non-transitory computer readable medium according to an embodiment will be described with reference to the accompanying drawings. In the embodiment(s) described below, elements assigned the same reference symbols are assumed to perform the same operations, and redundant descriptions thereof will be omitted as appropriate. Hereinafter, an embodiment will be described with reference to the accompanying drawings.

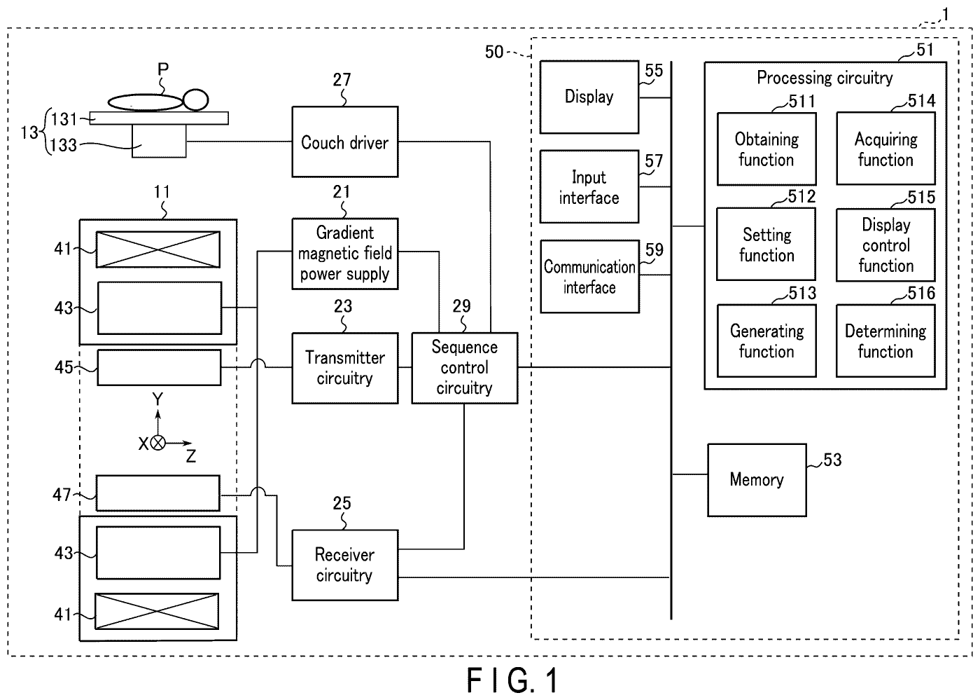

FIG. 1 is a block diagram showing an example of a configuration of a magnetic resonance data acquisition apparatus according to an embodiment. As shown in FIG. 1, a magnetic resonance data acquisition apparatus 1 is, for example, a magnetic resonance imaging apparatus. The magnetic resonance data acquisition apparatus 1 includes a gantry 11, a couch 13, a gradient magnetic field power supply 21, transmitter circuitry 23, receiver circuitry 25, a couch driver 27, sequence control circuitry 29, and a medical information processing apparatus (host computer) 50.

The gantry 11 includes a static magnetic field magnet 41 and a gradient magnetic field coil 43. The static magnetic field magnet 41 and the gradient magnetic field coil 43 are accommodated in a housing of the gantry 11. A bore with a hollow shape is formed in the housing of the gantry 11. A transmitter coil 45 and a receiver coil 47 are arranged in the bore of the gantry 11.

The static magnetic field magnet 41 has a hollow, approximately cylindrical shape, and generates a static magnetic field thereinside. For example, a permanent magnet, a superconducting magnet, or a normal conducting magnet is used as the static magnetic field magnet 41. Here, a central axis of the static magnetic field magnet 41 is defined as a Z-axis, an axis vertically orthogonal to the Z-axis is defined as a Y-axis, and an axis horizontally orthogonal to the Z-axis is defined as an X-axis. The X-axis, Y-axis, and Z-axis constitute an orthogonal three-dimensional coordinate system.

The gradient magnetic field coil 43 is a coil unit attached to the inside of the static magnetic field magnet 41 and formed in a hollow, approximately cylindrical shape. The gradient magnetic field coil 43 receives a supply of a current from the gradient magnetic field power supply 21 to generate a gradient magnetic field. More specifically, the gradient magnetic field coil 43 includes three coils corresponding to the X-axis, the Y-axis, and the Z-axis, which are orthogonal to each other. The three coils form gradient magnetic fields in which the magnetic field intensity varies along the X-axis, the Y-axis, and the Z-axis, respectively. The gradient magnetic fields along the X-axis, the Y-axis, and the Z-axis are combined to form, in desired directions, a frequency encoding gradient magnetic field Gr, a phase encoding gradient magnetic field Gp, and a slice selection gradient magnetic field Gs, which are orthogonal to each other. The frequency encoding gradient magnetic field Gr is used to change the frequency of a magnetic resonance signal (hereinafter, referred to as an “MR signal”) in accordance with a spatial position. The phase encoding gradient magnetic field Gp is used to change the phase of an MR signal in accordance with a spatial position. The slice selection gradient magnetic field Gs is used to discretionarily determine an imaging cross-section (slice). The following description is based on the premise that the gradient direction of the frequency encoding gradient magnetic field Gr aligns with the X-axis, the gradient direction of the phase encoding gradient magnetic field Gp aligns with the Y-axis, and the gradient direction of the slice selection gradient magnetic field Gs aligns with the Z-axis.

The gradient magnetic field power supply 21 supplies a current to the gradient magnetic field coil 43 in accordance with a sequence control signal from the sequence control circuitry 29. Through the supply of a current to the gradient magnetic field coil 43, the gradient magnetic field power supply 21 causes the gradient magnetic field coil 43 to generate gradient magnetic fields along the X-axis, the Y-axis, and the Z-axis. The gradient magnetic fields are superimposed on a static magnetic field formed by the static magnetic field magnet 41 and applied to a subject P.

The transmitter coil 45 is, for example, arranged inside the gradient magnetic field coil 43, and generates a high-frequency pulse (hereinafter, referred to as an “RF pulse”) upon receiving a current supplied from the transmitter circuitry 23.

The transmitter circuitry 23 supplies a current to the transmitter coil 45 in order to apply an RF pulse for exciting a target proton existing in the subject P to the subject P via the transmitter coil 45. The RF pulse oscillates at a resonance frequency specific to the target proton to excite the target proton. An MR signal is generated from the excited target proton and is detected by the receiver coil 47. The transmitter coil 45 is, for example, a whole-body coil (WB coil). The whole-body coil may be used as a transmitter-receiver coil.

In response to an action of the RF pulse, the receiver coil 47 receives the MR signal generated from the target proton in the subject P. The receiver coil 47 has a plurality of receiver coil elements capable of receiving an MR signal. The received MR signal is supplied to the receiver circuitry 25 either wirelessly or via a wire.

Although not shown in FIG. 1, the receiver coil 47 has a plurality of reception channels arranged in parallel. Each reception channel has a receiver coil element for receiving an MR signal, an amplifier for amplifying the MR signal, etc. An MR signal is output from each reception channel. The total number of reception channels may be equal to, larger than, or smaller than the total number of receiver coil elements.

The receiver circuitry 25 receives an MR signal generated from the excited target proton via the receiver coil 47. The receiver circuitry 25 processes the received MR signal to generate a digital MR signal. The digital MR signal can be expressed by a k-space defined by spatial frequency. Thus, digital MR signals are referred to as “k-space data”. The k-space data is an example of an MR acquisition signal. The k-space data is supplied to the medical information processing apparatus 50 either wirelessly or via a wire.

The transmitter coil 45 and the receiver coil 47 described above are mere examples. Instead of the transmitter coil 45 and the receiver coil 47, a transmitter-receiver coil having both a transmitting function and a receiving function may be used. Alternatively, the transmitter coil 45, the receiver coil 47, and a transmitter-receiver coil may be combined.

The couch 13 is installed adjacently to the gantry 11. The couch 13 has a top board 131 and a base 133. The subject P is placed on the top board 131. The base 133 supports the top board 131 slidably along each of the X-axis, the Y-axis, and the Z-axis. The couch driver 27 is accommodated in the base 133. The couch driver 27 moves the top board 131 under the control of the sequence control circuitry 29. The couch driver 27 may include, for example, any motor such as a servo motor or a stepping motor.

The sequence control circuitry 29 includes, as hardware resources, a processor such as a central processing unit (CPU) or a micro processing unit (MPU), and a memory such as a read only memory (ROM) or a random access memory (RAM). The sequence control circuitry 29 controls the gradient magnetic field power supply 21, the transmitter circuitry 23, and the receiver circuitry 25 synchronously based on data acquisition conditions set by the processing circuitry 51, subjects the subject P to data acquisition corresponding to the data acquisition conditions, and acquires k-space data relating to the subject P. The sequence control circuitry 29 is an example of a sequence controller.

The sequence control circuitry 29 according to the present embodiment carries out general data acquisition for MR images and data acquisition for magnetic resonance spectroscopy (MRS), which is a type of chemical shift measurement. Since the data acquisition for MR images is a general technique, a detailed description thereof will be omitted. The chemical shift measurement is a technique of measuring a chemical shift, which is a minor difference in resonance frequency of a target proton such as a hydrogen nucleus, which is caused in accordance with a difference in chemical environment. The MRS includes a single voxel method in which data acquisition is performed on a single voxel and a multi-voxel method in which data acquisition is performed on multiple voxels, and the present embodiment can be applied to either method. The multi-voxel method is also referred to as chemical shift imaging (CSI), MRS imaging (MRSI), or the like. The voxel of a measurement-target region is also referred to as “a voxel of interest (VOI).” Also, in the present embodiment, a region including a voxel of interest is referred to as “a region of interest (ROI).”

The sequence control circuitry 29 performs data acquisition for MRS on the subject P. When the data acquisition for MRS is performed, a free induction decay (FID) signal or a spin echo signal is generated from the voxel of interest of the subject P. The receiver circuitry 25 receives an FID signal or a spin echo signal via the receiver coil 47, and processes the received FID signal or spin echo signal to acquire k-space data related to the voxel of interest. Let us assume that the acquired k-space data is digital data expressing, by a time function, the value of the intensity of the signal emitted from the voxel of interest. A pulse sequence for MRS is repeated for the number of excitations (NEX), and k-space data corresponding to the number of excitations is acquired. Hereinafter, the k-space data acquired through MRS will be referred to as “MRS k data.” The MRS k data is an example of an MRS signal.

In the present embodiment, the MRS pulse sequence may be a pulse sequence used to acquire an MRS signal, such as a LASER (localization by adiabatic selective refocusing) method, an ISIS method, a semi-LASER method, and a PRESS method.

As shown in FIG. 1, the medical information processing apparatus 50 is a computer that includes processing circuitry 51, a memory 53, a display 55, an input interface 57, and a communication interface 59.

The processing circuitry 51 has a processor such as a CPU as a hardware resource. The processing circuitry 51 functions as the main unit of the magnetic resonance data acquisition apparatus 1. For example, the processing circuitry 51 executes various programs to implement an obtaining function 511, a setting function 512, a generating function 513, an acquiring function 514, a display control function 515, and a determining function 516.

By implementing the obtaining function 511, the processing circuitry 51 obtains a designated region of interest.

By implementing the setting function 512, the processing circuitry 51 sets an acquisition method for acquiring multiple pieces of magnetic resonance data.

By implementing the generating function 513, the processing circuitry 51 generates a pulse sequence for acquiring magnetic resonance data multiple times based on the region of interest while changing at least one of a region for acquisition or a condition of setting a saturation pulse.

By implementing the acquiring function 514, the processing circuitry 51 acquires multiple pieces of magnetic resonance data according to the pulse sequence.

By implementing the display control function 515, the processing circuitry 51 causes the multiple pieces of magnetic resonance data to be displayed, for example, on the display 55.

By implementing the determining function 516, the processing circuitry 51 determines, from the multiple pieces of magnetic resonance data, a condition of setting a saturation pulse or a region of interest that makes an influence of a strong peak signal in an acquisition-target region equal to or below a threshold. Examples of the strong peak signal include a fat signal, a signal that is mixed in from the outside of the acquisition-target region, etc.

The memory 53 is a storage device such as a hard disk drive (HDD), a solid state drive (SSD), or an integrated circuit storage device for storing various kinds of information. The memory 53 may be, for example, a drive that reads and writes various kinds of information from and to a portable storage medium such as a CD-ROM drive, a DVD drive, or a flash memory. For example, the memory 53 stores medical data acquired in the past, an MRS signal, a control program, etc.

The display 55 displays various kinds of information. For example, a CRT display, a liquid crystal display, an organic EL display, an LED display, a plasma display, or any other display known in the relevant technical field can be suitably used as the display 55.

The input interface 57 includes an input device that receives various commands from a user. Examples of the input device that can be used include a keyboard, a mouse, various switches, a touch screen, and a touch pad. The input device is not limited to a device equipped with physical operational parts such as a mouse and a keyboard. Examples of the input interface 57 also include electric signal processing circuitry that receives an electric signal corresponding to an input operation from an external input device provided separately from the magnetic resonance data acquisition apparatus 1, and outputs the received electric signal to various types of circuitry. The input interface 57 may also be a voice recognition device that collects voice signals via a microphone and converts the voice signals into command signals.

The communication interface 59 is an interface that connects the magnetic resonance data acquisition apparatus 1 with a workstation, a picture archiving and communication system (PACS), a hospital information system (HIS), a radiology information system (RIS), etc., via a local area network (LAN), etc. The communication interface 59 transmits and receives various kinds of information to and from the connected workstation, PACS, HIS, and RIS.

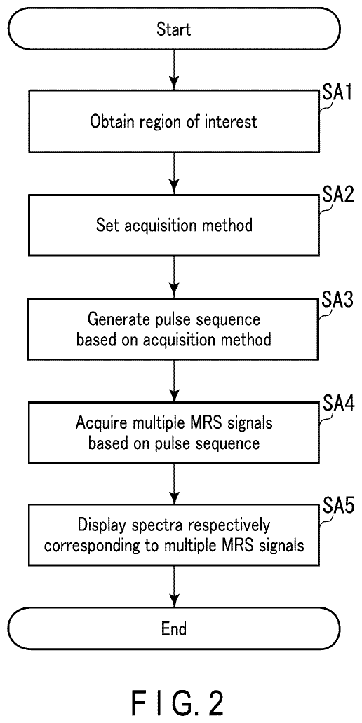

Next, an example of an operation of the magnetic resonance data acquisition apparatus 1 according to the embodiment will be described with reference to the FIG. 2. The example of the operation shown in FIG. 2 assumes a case where data based on an MRS signal is used as magnetic resonance data and assumes a case where a process prior to the main acquisition of an MRS signal is performed. Specifically, it is a process for determining a condition of setting a saturation pulse or a region of interest, and in this process, a condition of setting a saturation pulse or a position of a region of interest in which an influence of a strong peak signal is reduced is determined. Hereinafter, a case of reducing an influence of fat will be assumed as an example of reducing an influence of a strong peak signal. It is also assumed herein that an MR image of a subject P is obtained in advance, and that a region of interest is designated on the MR image.

In step SA1, the processing circuitry 51 obtains a designated region of interest by implementing the obtaining function 511. For example, a user may set a desired region of interest on a captured MR image of a subject P.

Alternatively, the processing circuitry 51 may set, in advance, a region of interest by referring to the case of the subject P or the past imaging history of the subject P.

In step SA2, the processing circuitry sets an acquisition method by implementing the setting function 512. For example, the acquisition method may be set by designation by a user. Specifically, the following is set: whether a first method is adopted in which a region for acquisition is changed and data is acquired multiple times, a second method is adopted in which a saturation pulse around a region of interest is changed and data is acquired multiple times, or both the first method and the second method are adopted.

For example, if a part of the brain that is close to the scalp is set as the region of interest, the first method can be used to perform acquisition while searching for a position for acquisition in which an MRS signal with the least fat can be obtained while shifting the region for acquisition to the central side of the head, since it is considered that a fat signal is likely to be mixed in a spectrum of a metabolite. Alternatively, if the position of the region of interest obtained in step SAI is to be respected, the second method can be adopted to determine what saturation pulse should be set in order to acquire an MRS signal in the region of interest.

A method that is adopted by default or a method that is adopted based on the position of the region of interest or the information on the case may be set. In the case of the head, for example, setting may be made such that the first method is adopted.

In step SA3, the processing circuitry 51 generates a pulse sequence based on the acquisition method by implementing the generating function 513. Step SA3 is a step of generating a sequence for acquiring MRS signals multiple times in order to determine an influence of fat as pre-scanning. Thus, a pulse sequence is designed such that the time for acquiring MRS signals becomes short, such as setting the number of excitations (NEX) to “1.” For example, a general repetition time (TR) for a pulse sequence relating to the main acquisition is about 2000 ms, but the repetition time (TR) herein may be set to, for example, as short as about 500 to 800 ms.

Also, a water-suppression pulse need not be incorporated into the pulse sequence; if it is to be incorporated into the pulse sequence, it may be simplified as compared to the case of the main acquisition. For example, rather than applying a number of pulses, such as WET (water suppression enhanced through T1 effects) and VAPOR (variable pulse power and optimized relaxation delays), one or two pulses may be applied.

Alternatively, the band of the water-suppression pulse may be broadened. For example, if a band of a spectrum that is to be suppressed by the water-suppression pulse is usually 1 ppm, the band may be changed to a broader band of 2 ppm.

Also, for example, the readout time may be set to a half or ¾ of the time as compared to the case of the main acquisition. That is, any method may be adopted, provided that TR becomes short.

In step SA4, by implementing the acquiring function 514, the processing circuitry 51 acquires magnetic resonance data corresponding to acquisition performed multiple times, herein, multiple MRS signals, according to the pulse sequence generated in step SA3.

In step SA5, by implementing the display control function 515, the processing circuitry 51 causes spectra respectively corresponding to the multiple MRS signals acquired to be displayed. A corresponding region for acquisition or condition for acquisition may also be displayed together with the spectra.

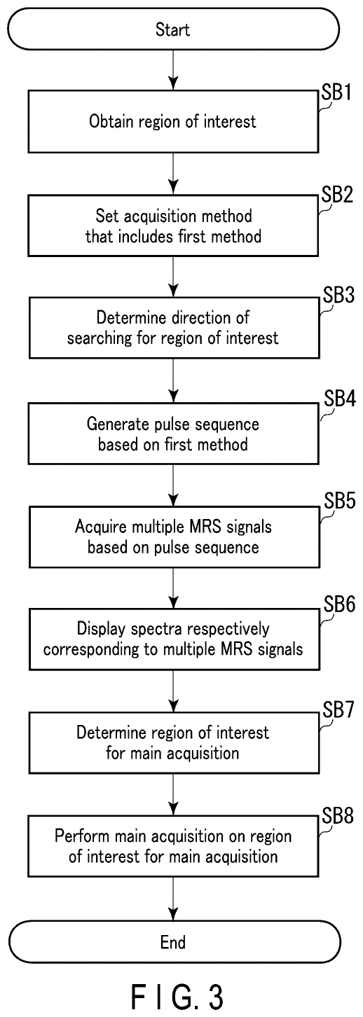

Next, a detailed example of an operation performed by the magnetic resonance data acquisition apparatus 1 with an acquisition method that includes the first method will be described with reference to the flowchart shown in FIG. 3.

In step SB1, the processing circuitry 51 obtains a designated region of interest by implementing the obtaining function 511.

In step SB2, the processing circuitry 51 sets an acquisition method that includes the first method by implementing the setting function 512.

In step SB3, the processing circuitry 51 determines a direction of searching for a region of interest by implementing the setting function 512. The searching direction may be determined by a user designating the searching direction or through automatic estimation. If the searching direction is to be determined through automatic estimation, the processing circuitry 51 may, for example, estimate a direction perpendicular to the contour of the region of interest by using a trained model, pattern matching, etc., and determine it as the searching direction. Specifically, the following may be performed:

input the region of interest and estimate a direction perpendicular to the contour of the region of interest using a model trained to estimate a direction perpendicular to the region of interest or estimate a direction perpendicular to the contour of the region of interest through pattern matching.

In step SB4, the processing circuitry 51 sets a pulse sequence based on the first method by implementing the generating function 513. That is, a pulse sequence for performing acquisition multiple times while shifting the region of interest along the searching direction is set.

In step SB5, the processing circuitry 51 acquires an MRS signal for each region of interest according to the pulse sequence by implementing the acquiring function 514.

In step SB6, by implementing the display control function 515, the processing circuitry 51 causes spectra respectively corresponding to the multiple MRS signals acquired to be displayed.

In step SB7, by implementing the determining function 516, the processing circuitry 51 determines a region of interest desired by a user among multiple regions for acquisition to be a region of interest for the main acquisition according to the user's instruction. Specifically, by implementing the display control function 515, for example, the processing circuitry 51 causes multiple regions of interest and corresponding MRS signals to be displayed on the display 55. The user selects a desired region of interest. By implementing the determining function 516, the processing circuitry 51 may obtain the user's selection as a user's instruction and determine the selected region of interest to be a region of interest for the main acquisition. Alternatively, by implementing the determining function 516, the processing circuitry 51 may select, as a region of interest for the main acquisition, a region of interest corresponding to a spectrum that is least influenced by fat. For example, the position of the region of interest corresponding to a spectrum having a peak value of fat that is equal to or below a threshold may be determined to be a region of interest for the main acquisition.

In step SB8, by implementing the acquiring function 514, the processing circuitry 51 can perform the main acquisition on the region of interest for the main acquisition and acquire an MRS signal with reduced fat. Note that a method of displaying an MRS signal-based spectrum is not limited to displaying spectra that are based on respective MRS signals. For example, by implementing the determining function 516, the processing circuitry 51 selects, as the region of interest for the main acquisition, one region of interest in which fat is most suppressed and a desired signal is acquired among multiple regions for acquisition. In this case, step SB6 may be omitted, and with the display control function 515, the processing circuitry 51 may cause the selected one region of interest for the main acquisition to be displayed.

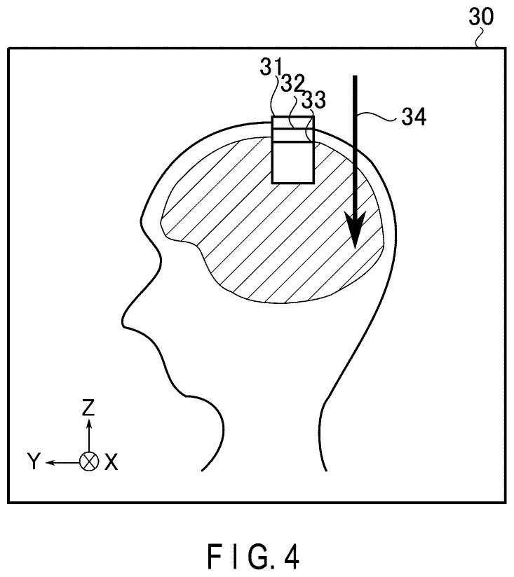

Next, an example of setting a region for acquisition according to the first method will be described with reference to FIG. 4.

FIG. 4 shows an example in which regions for acquisition including multiple regions of interest are set on an MR image 30 of a subject P. Multiple MRS signals are acquired while shifting the regions of interest every 1 TR along a searching direction 34. For example, if a region of interest 31 is designated at the top of the head, basically, MRS signals may be acquired by switching the regions of interest while changing the frequency shift of the slice selection pulse (z-axis direction) every 1 TR with the three regions of interest 31 to 33 set as the regions for acquisition in the example shown in FIG. 4. By implementing the setting function 512, the processing circuitry 51 may set multiple regions for acquisition in the y-axis direction and the x-axis direction as searching directions.

Herein, the example is shown in which multiple regions are set as the regions for acquisition along the searching direction 34, starting from the region of interest 31 toward the inner side of the head; however, multiple regions may be set in a direction toward the outside of the head or set in the top-to-bottom direction (front-to-back direction) with respect to the regions of interest. For example, if the region of interest 33 is a designated region of interest, the region of interest 32 and the region of interest 31 may be set as the regions for acquisition in an outer direction of the head.

Also, the searching direction 34 is not limited to a direction perpendicular to the set region of interest, and may be an oblique direction or a rotational direction. For example, it is possible to set a region of interest in such a manner as to surround a periphery of a region of interest and set a region for acquisition. That is, setting of a region may be performed so as to enable searching for a better candidate region of interest around a designated region of interest.

Shimming adjustment values vary depending on the positions of the regions of interest 31 to 33. For example, the shimming value may be adjusted based on a region of interest considered to be less influenced by fat among multiple regions for acquisition. In FIG. 4, the region of interest 33 is less influenced by fat, as compared to the region of interest 31; thus, the shimming value may be adjusted based on the region of interest 33.

Alternatively, the shimming value may be adjusted based on the regions for acquisition as a whole. That is, in FIG. 4, the shimming value may be adjusted based on the totality of the regions of interest 31 to 33. Furthermore, a method of adjusting a shimming value more precisely may be calculating a shimming value for each region for acquisition and adjusting it with a corresponding shimming value when acquiring an MRS signal from a target region of interest. That is, in FIG. 4, a shimming value may be calculated in advance in each of the regions of interest 31 to 33 and adjusted by referring to a corresponding shimming value at the time of acquisition.

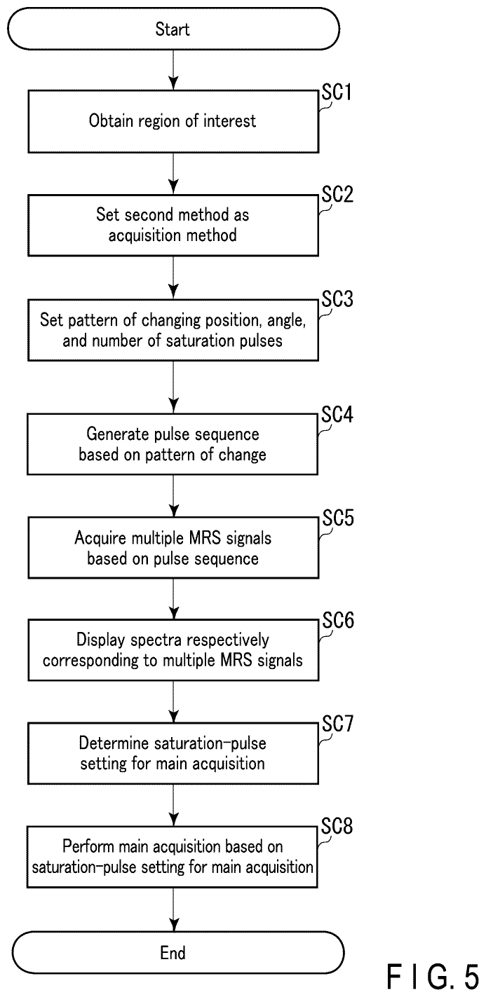

Next, a detailed example of an operation performed by the magnetic resonance data acquisition apparatus 1 with an acquisition method that includes the second method will be described with reference to the flowchart shown in FIG. 5.

In step SC1, the processing circuitry 51 obtains a designated region of interest by implementing the obtaining function 511.

In step SC2, by implementing the setting function 512, the processing circuitry 51 sets an acquisition method that includes the second method.

In step SC3, by implementing the setting function 512, the processing circuitry 51 sets a pattern of change relating to the position, angle, and number of saturation pulses for a region of interest. For example, in the case of changing the position of a saturation pulse, a pattern may be set in which the position of the saturation pulse is set on the right side of the region of interest in the first acquisition and the position of the saturation pulse is set on the left side of the region of interest in the second acquisition. Likewise, in the case of changing the angle of a saturation pulse, the change may be set as an angle of 30 degrees in the first acquisition and an angle of 45 degrees in the second acquisition. In the case of changing the number of saturation pulses, the change may be made as two saturation pulses in the first acquisition and three saturation pulses in the second acquisition. As a matter of course, the changes in the position, angle, and number of saturation pulses may be combined.

In step SC4, the processing circuitry 51 generates a pulse sequence based on the pattern of change by implementing the generating function 513.

In step SC5, by implementing the acquiring function 514, the processing circuitry 51 acquires multiple MRS signals based on the pulse sequence, that is, while changing respective conditions of a saturation pulse according to the pattern of change in the saturation pulse.

In step SC6, by implementing the display control function 515, the processing circuitry 51 causes spectra based on the multiple MRS signals acquired to be displayed.

In step SC7, by implementing the determining function 516, the processing circuitry 51 determines a condition desired by a user among the respective conditions in the pattern of change to be saturation-pulse setting for the main acquisition according to the user's instruction. Specifically, by implementing the display control function 515, for example, the processing circuitry 51 causes the respective conditions in the pattern of change and corresponding spectra to be displayed on the display 55.

The user selects a desired pattern of change in the saturation pulse. By implementing the determining function 516, the processing circuitry 51 may obtain the selection as the user's instruction and determine the selected pattern of change in the saturation pulse to be saturation-pulse setting for the main acquisition. Alternatively, by implementing the determining function 516, the processing circuitry 51 may select, as saturation-pulse setting for the main acquisition, a condition of a saturation pulse with which a spectrum that is least influenced by fat is obtained. That is, a condition of a saturation pulse corresponding to a spectrum having a peak value of fat that is equal to or below a threshold may be selected as saturation-pulse setting for the main acquisition. As the criterion of setting a saturation pulse, a saturation pulse may be set in a region that does not deviate from and is as close as possible to an intended region of interest. Also, a condition may be set in which mixing of a fat signal in a region of interest is suppressed to the extent possible and there are few saturation pulses.

In step SC8, by implementing the acquiring function 514, the processing circuitry 51 applies a saturation pulse based on the saturation-pulse setting for the main acquisition to perform the main acquisition according to a pulse sequence related to the main acquisition, and acquires an MRS signal.

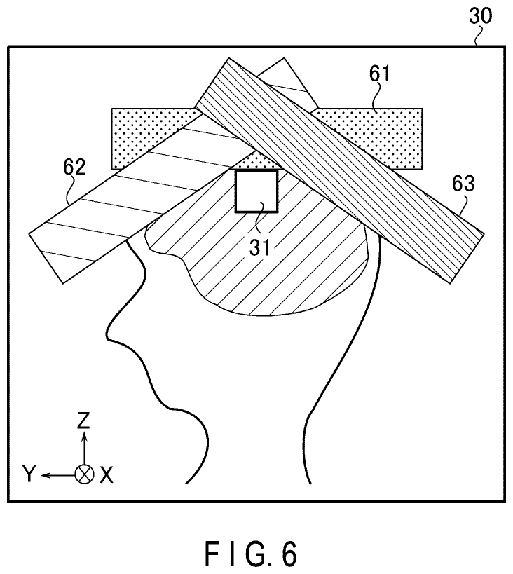

Next, an example of setting a pattern of change in a saturation pulse according to the second method will be described with reference to FIG. 6.

FIG. 6 shows an example in which the region of interest 31 is set on the MR image 30 of the subject P and saturation pulses 61 to 63 are set around the region of interest 31. Herein, saturation pulses, a condition of applying one saturation pulse (the saturation pulse 61), a condition of applying two saturation pulses (the saturation pulses 61 and 62), and a condition of applying three saturation pulses (the saturation pulses 61, 62, and 63) are set as patterns of change. Since it is desired that the acquisition time be short in the main acquisition as well, a condition with the least number of saturation pulses among the conditions of saturation pulses in which fat is suppressed, that is, the influence of fat on the spectra is equal to or below a threshold, may be determined to be the saturation-pulse setting for the main acquisition by referring to spectra that are based on MRS data acquired under each condition.

If both the first method and the second method are included in the acquisition method, the processes shown in FIGS. 3 and 5 may be performed in combination. For example, the following combination may be carried out: the saturation pulse 61 is set in the first acquisition at the regions of interest 31 to 33 shown in FIG. 4 and the saturation pulses 61 and 62 are set in the second acquisition at the regions of interest 31 to 33.

The above example assumes a case where a single voxel is set as a region of interest via a single voxel method, but the same process can be applied even when acquisition is carried out via a multi-voxel method relating to multiple voxels.

In a multi-voxel method, since a region of interest is in a field of view (FOV), magnetic resonance data may be acquired multiple times based on the region of interest in the FOV while changing at least one of a region for acquisition or a saturation pulse as described above. Also, multiple pieces of magnetic resonance data for determining fat as described above may be acquired based on the entire region in the FOV.

Furthermore, multiple pieces of magnetic resonance data for determining fat as described above may be acquired based on a region having a size between the size of the FOV and the size of the region of interest. For example, if the FOV has a voxel size of 12×12 and the region of interest has a voxel size of 4×4 with respect to a single voxel size of 1×1, the acquisition may be based on a region having a voxel size of 8×8 as a middle size. Also, in the case of performing acquisition via a multi-voxel method, the above-mentioned determination may be performed based on any region in the FOV.

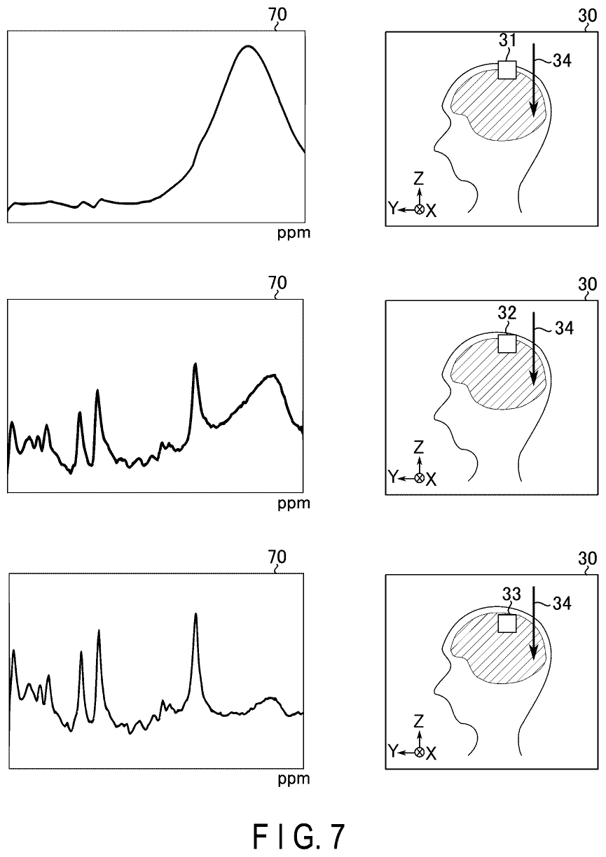

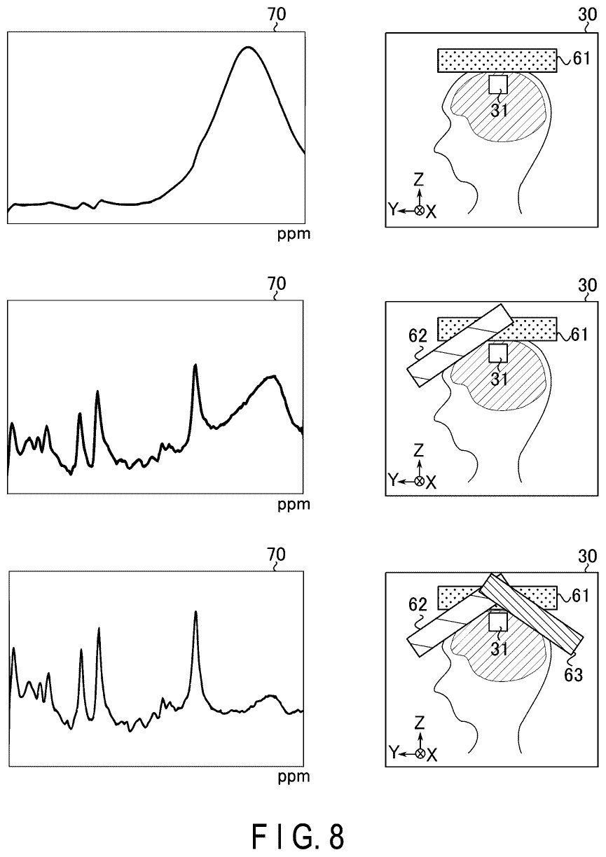

Next, an example of display through the display control function 515 will be described with reference to FIGS. 7 and 8.

FIG. 7 shows an example of displaying spectra of multiple MRS signals based on the acquisition method with the first method. The figures on the left side show spectra 70 based on MRS signals, and the figures on the right side show the positions of the regions of interest set on the MR images 30 corresponding to the spectra 70. Herein, the positions of the regions of interest 31 to 33 are displayed in association with three spectra, respectively. The spectra 70 and the MR images 30 in which the regions of interest are designated may be displayed on different windows. A user can select the position of a desired region of interest.

The maximum number of times of acquiring MRS signals and the number of times of acquisition indicating how many times the acquisition has been carried out up to the current stage may be displayed in association with the spectra 70 or the MR images 30 that include the regions of interest.

Also, the searching directions and the amounts of shift from the regions of interest as a reference may be displayed in association with the spectra 70 or the MR images 30 that include the regions of interest.

Alternatively, by implementing the display control function 515, the processing circuitry 51 may cause one spectrum 70 and the MR image 30 corresponding thereto to be displayed on a screen and cause each region of interest or a spectrum of each acquisition condition to be displayed dynamically by, for example, scrolling with a mouse, such that they can be switched. This allows the user to dynamically check the change in the spectrum corresponding to the change in the acquisition condition or the position of the region of interest.

The MR images 30 displayed may be cross-sectional images in which the regions of interest are set. Since an influence of fat on a spectrum is determined in the present embodiment, if a region of interest is designated at the top of the head, for example, an MR image of a sagittal section may be displayed, and if parts of the left and right brains in the head region are designated, for example, an MR image of a coronal section or a horizontal section may be displayed.

As shown in FIG. 7, spectra 70 obtained vary depending on the regions of interest. For example, in the upper figure of FIG. 7, a fat tissue around the region of interest is excited, and there is a large influence of the fat, making it impossible to detect a spectrum of a metabolite. On the other hand, in the lower figure of FIG. 7, there is little influence of the fat, making it possible to visually recognize a spectrum of a metabolite. A user can determine the region of interest corresponding to a spectrum with no fat mixed in to be the region of interest for the main acquisition.

Next, FIG. 8 shows an example of displaying multiple pieces of MR data based on the acquisition method with the second method.

Spectra 70 based on MRS data are shown on the left side of the figure, and conditions of setting saturation pulses corresponding to the spectra are shown on the right side of the figure and are displayed on the MR images as the saturation pulses 61 to 63 in association with the spectra. Herein, the conditions of saturation pulses are displayed in association with three spectra 70, respectively. As in the case of FIG. 7, a user can select optimum saturation-pulse setting for the main acquisition with regard to the saturation pulses by referring to the spectra 70.

The spectra and the regions of interest or the conditions of saturation pulses shown in FIGS. 7 and 8 may be displayed in real time by being switched each time acquisition is performed. For example, by implementing the display control function 515, the processing circuitry 51 causes the spectrum 70 in the upper figure of FIG. 7 subjected to image processing to be displayed on a screen after the acquisition of the MRS signal shown in the upper figure of FIG. 7 is completed. Once the acquisition of the next MRS signal is completed, the display is switched to the spectrum 70 in the middle figure of FIG. 7.

If a user determines that a desired spectrum has been obtained when the spectra are displayed in real time, the acquisition of MR data may be terminated in the middle. For example, if a desired spectrum is being displayed, a currently displayed region of interest or condition of a saturation pulse may be determined to be a region of interest for the main acquisition or saturation-pulse setting for the main acquisition by pressing or clicking a predetermined button. Also, for example, if the magnetic resonance data acquisition apparatus automatically determines a region of interest for the main acquisition or saturation-pulse setting for the main acquisition, a region of interest or a saturation pulse corresponding to a spectrum in which a peak of fat is equal to or below one-tenth of a peak value of a metabolite NAA may be selected. With the processing circuitry 51 causing a spectrum relating to selection to be displayed by implementing the display control function 515, a user can easily visually recognize the shape of the spectrum and can thus confirm the spectrum based on which the determination is made.

The above-described example assumes a case where magnetic resonance data is data based on an MRS signal; however, the embodiment is not limited thereto. For example, the determination process performed by the magnetic resonance data acquisition apparatus according to the present embodiment can also be used in a CEST (chemical exchange saturation transfer) method in which an image of trace amounts of molecules is formed by utilizing the phenomenon of proton exchange between intramolecular protons and in vivo bulk water protons, in order to achieve the effect of suppressing fat signals.

According to the embodiment described above, the generating unit generates a pulse sequence for acquiring magnetic resonance data multiple times based on a region of interest while changing at least one of a region for acquisition or a condition of setting a saturation pulse. The acquiring unit acquires multiple pieces of magnetic resonance data according to the pulse sequence. Thus, it is possible to determine, from the spectra based on multiple pieces of magnetic resonance data, a condition of setting a saturation pulse and a position of a region of interest where mixing of fat signals in the magnetic resonance data is reduced that can be used in the main acquisition. Whether the determination is made by a user or automatically by an apparatus, the positions of multiple regions of interest or conditions of setting saturation pulses can be determined through comparison by referring to spectra; thus, it is possible to reduce the influence of fat signals on spectra with high precision while reducing the dependency on an operator.

The terminology “processor” used in the above description refers to, for example, circuitry such as a CPU (central processing unit), a GPU (graphics processing unit), an ASIC (application specific integrated circuit), a programmable logic device (such as a simple programmable logic device (SPLD), a complex programmable logic device (CPLD), and a field programmable gate array (FPGA)), etc. If the processor is, for example, a CPU, the processor implements the functions by reading and executing programs stored in storage circuitry. On the other hand, if the processor is an ASIC, for example, its functions are directly incorporated into the circuitry of the processor as logic circuitry, instead of a program being stored in the storage circuitry. The processors described in connection with the above embodiments are not limited to single-circuit processors; a plurality of independent processors may be integrated into a single processor that implements the functions of the processors. Furthermore, the functions may be implemented by a single processor into which multiple components shown in the drawings are incorporated.

In addition, the functions described in the above embodiment may be implemented, for example, by installing programs for executing the processing in a computer, such as a workstation, and expanding the programs in a memory. The programs that can cause the computer to execute the processing can be stored in a storage medium, such as a magnetic disk (a hard disk, etc.), an optical disk (CD-ROM, DVD, etc.), or a semiconductor memory and distributed through it.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A magnetic resonance data acquisition apparatus comprising processing circuitry configured to:
obtain a designated region of interest;
generate a pulse sequence for acquiring magnetic resonance data multiple times based on the region of interest while changing at least one of a region for acquisition or a condition of setting a saturation pulse; and
acquire multiple pieces of magnetic resonance data according to the pulse sequence.

2. The magnetic resonance data acquisition apparatus according to claim 1, wherein the processing circuitry is further configured to determine, from the multiple pieces of magnetic resonance data, a condition of setting a saturation pulse or a region of interest that makes an influence of fat in an acquisition-target region equal to or below a threshold.

3. The magnetic resonance data acquisition apparatus according to claim 1, wherein the processing circuitry is further configured to perform control so that (a) the at least one of the region for acquisition or the condition of setting a saturation pulse changed by the processing circuitry and (b) corresponding magnetic resonance data are displayed; and
determine a condition of setting a saturation pulse or a region of interest that makes an influence of a strong peak signal in an acquisition-target region equal to or below a threshold based on one region for acquisition or one condition of setting a saturation pulse selected by a user.

4. The magnetic resonance data acquisition apparatus according to claim 1, wherein the processing circuitry is configured to generate the pulse sequence for acquiring magnetic resonance data multiple times while shifting a position of the region of interest along a designated direction, and
the multiple pieces of magnetic resonance data are data corresponding to respective positions of the region of interest changed.

5. The magnetic resonance data acquisition apparatus according to claim 1, wherein the processing circuitry is configured to generate the pulse sequence for acquiring magnetic resonance data multiple times while changing at least one condition among a position, an angle, and number of the saturation pulse, and
the multiple pieces of magnetic resonance data are data corresponding to respective conditions of the saturation pulse changed.

6. The magnetic resonance data acquisition apparatus according to claim 1, wherein if acquiring magnetic resonance data multiple times, the processing circuitry is configured to generate a pulse sequence having a repetition time for acquiring the magnetic resonance data multiple times that is shorter than a repetition time for main acquisition.

7. A magnetic resonance data acquisition method, comprising:
obtaining a designated region of interest;
generating a pulse sequence for acquiring magnetic resonance data multiple times based on the region of interest while changing at least one of a region for acquisition or a condition of setting a saturation pulse; and
acquiring multiple pieces of magnetic resonance data according to the pulse sequence.

8. The magnetic resonance data acquisition method according to claim 7, wherein further comprising determining, from the multiple pieces of magnetic resonance data, a condition of setting a saturation pulse or a region of interest that makes an influence of fat in an acquisition-target region equal to or below a threshold.

9. The magnetic resonance data acquisition method according to claim 7, wherein further comprising performing control so that (a) the at least one of the region for acquisition or the condition of setting a saturation pulse changed by the processing circuitry and (b) corresponding magnetic resonance data are displayed; and
determining a condition of setting a saturation pulse or a region of interest that makes an influence of a strong peak signal in an acquisition-target region equal to or below a threshold based on one region for acquisition or one condition of setting a saturation pulse selected by a user.

10. The magnetic resonance data acquisition method according to claim 7, wherein the generating the pulse sequence generates the pulse sequence for acquiring magnetic resonance data multiple times while shifting a position of the region of interest along a designated direction, and
the multiple pieces of magnetic resonance data are data corresponding to respective positions of the region of interest changed.

11. The magnetic resonance data acquisition method according to claim 7, wherein the generating the pulse sequence generates the pulse sequence for acquiring magnetic resonance data multiple times while changing at least one condition among a position, an angle, and number of the saturation pulse, and
the multiple pieces of magnetic resonance data are data corresponding to respective conditions of the saturation pulse changed.

12. The magnetic resonance data acquisition method according to claim 7, wherein if acquiring magnetic resonance data multiple times, the generating a pulse sequence generates a pulse sequence having a repetition time for acquiring the magnetic resonance data multiple times that is shorter than a repetition time for main acquisition.

13. A non-transitory computer readable medium including computer executable instructions, wherein the instructions, when executed by a processor, cause the processor to perform a method comprising:
obtaining a designated region of interest;
generating a pulse sequence for acquiring magnetic resonance data multiple times based on the region of interest while changing at least one of a region for acquisition or a condition of setting a saturation pulse; and
acquiring multiple pieces of magnetic resonance data according to the pulse sequence.
